(19) United States
(12) Patent Application Publication
(10) Pub. No.: US 2007/0055013 A1
(43) Pub. Date: Mar. 8, 2007

(54) Substrate and method of immobilizing protein

(76) Inventors: Noriho Kamiya, Fukuoka City (JP); Masahiro Goto, Fukuoka City (JP); Satoshi Doi, Fukuoka City (JP); Koichiro Nakamura, Tokyo (JP); Noriyuki Kawata, Tokyo (JP); Koji Fujita, Tokyo (JP); Koki Tanamura, Tokyo (JP)

Correspondence Address: RATNERPRESTIA, P O BOX 980, VALLEY FORGE, PA, 19482-0980, US

(21) Appl. No.: 11/358,438

(22) Filed: Feb. 21, 2006

(30) Foreign Application Priority Data
Feb. 21, 2005 (JP) 2005-44,524

(51) Int. Cl. A61K 47/48

(52) U.S. Cl. 525/54.1

(57) ABSTRACT

A substrate for the immobilization of proteins is made by treating a carrier with an amino group-containing silicon compound represented by the following general formula:
(RO)₃Si—(CH₂)ₖ—(C₆H₄)₁—(CH₂)ₘ—(NHCH₂CH₂)ₙ—NH₂ (wherein R is an alkyl group, and k is 1, 2, 3 . . . , l is 0, 1, 2, 3 . . . , m is 0, 1, 2, 3 . . . , and n is 1, 2, 3 . . . ).

### BACKGROUND OF THE INVENTION

1. Field of the Invention

The present invention relates to a substrate, particularly a substrate for immobilizing a protein, and a method of immobilizing a protein on the substrate.

2. Related Art

Now, various functional proteins are utilized in the field related to biotechnology such as a protein array or the like. A recent progress in the field of the protein array is largely caused by the developments of surface chemistry, protein immobilization, protein labeling and detection thereof, and so on. Particularly, the protein immobilization is very important because it is largely dependent on a type of proteins to be immobilized different from DNA array. The proteins must be immobilized without losing the activity and with maintaining the activity in water as far as possible. In order to progress the technology of the protein array, it is essential to establish the technology of immobilizing the proteins.

In the protein array, the proteins are most often utilized by immobilizing on a carrier. In general, a glass slide, a porous gel, a microtiter prate and the like are used as a carrier for immobilization. Also, when the carrier for immobilization is the glass slide, there is known a slide glass in which an amino group is introduced onto the surface thereof. Moreover, as a method of immobilizing proteins to the amino groups on the carrier, there are known a method in which the amino group is activated by glutaraldehyde and bonded to a terminal amino group of the proteins, and the like.

JP-A-2004-093330 discloses a DNA reaction detection chip, wherein an activated molecule having an amino group in its terminal is introduced onto the surface of the slide glass. JP-A-2003-161731 discloses a substrate for a microchip, wherein an amino group is introduced onto a surface of a plastic substrate by an aminoalkyl silane.

However, the methods as disclosed above have the following problem. More specifically, in the case of a slide glass having an aminopropyl group introduced onto the surface, there is a problem that a force of immobilizing proteins is weak and an amount of immobilized proteins decreases when the slide is used repeatedly.

### SUMMARY OF THE INVENTION

It is, therefore, an object of the present invention to solve the above problem and to provide an aminated substrate for the immobilization of proteins capable of strongly immobilizing the proteins and maintaining the activity of the proteins even in the repetitive use.

The inventors have made various studies in order to achieve the above object, and found out a substrate and a method of immobilizing proteins according to the present invention.

The substrate according to the present invention is characterized by treating a carrier with an amino group-containing silicon compound represented by the following general formula:
(RO)₃Si—(CH₂)ₖ—(C₆H₄)₁—(CH₂)ₘ—(NHCH₂CH₂)ₙ—NH₂
(wherein R is an alkyl group, and k is 1, 2, 3 . . . l is 0, 1, 2, 3 . . . , m is 0, 1, 2, 3 . . . , n is 1, 2, 3 . . . ) to introduce an amino group onto a surface of the carrier.

In a preferable embodiment of the substrate according to the present invention, n is 3-20.

In another preferable embodiment of the substrate according to the present invention, the amino group-containing silicon compound is (3-trimethoxysilylpropyl)-diethylene triamine.

Furthermore, the present invention is a method of immobilizing proteins to a substrate, characterized in that the substrate is made by treating a carrier with an amino group-containing silicon compound represented by the following general formula:
(RO)₃Si—(CH₂)ₖ—(C₆H₄)₁—(CH₂)ₘ—(NHCH₂CH₂)ₙ—NH₂
(wherein R is an alkyl group, and k is 1, 2, 3 . . . , l is 0, 1, 2, 3 . . . , m is 0, 1, 2, 3 . . . , n is 1, 2, 3 . . . ) to introduce an amino group onto a surface of the carrier, and immersed in a protein-containing buffer solution to immobilize the protein to the amino group through electrostatic interaction.

In a preferable embodiment of the method according to the present invention, the buffer solution contains a protein, a biopolymer or a biological sample (a cell, a microorganism, a tissue, an organ, etc.).

In another preferable embodiment of the method of the present invention, when l in the general formula is more than 1, the protein is immobilized through hydrophobic interaction of phenyl group.

In the other preferable embodiment of the method according to the present invention, when an isoelectric point of the protein is pI and an acid dissociation constant of the amino group is pKa, a pH of the protein-containing buffer solution satisfies a relation of pKa>pH>pI in the immobilization of the protein.

The present invention has an advantage that the force of immobilizing the proteins can be improved but also the activity of the proteins can be maintained even in the repetitive use.

### BRIEF DESCRIPTION OF THE DRAWINGS

FIG. 1 is a view illustrating a relation among pKa of an amino group, pI of a protein and pH of a protein-containing buffer solution.

### BEST MODE FOR CARRYING OUT THE INVENTION

The substrate of the present invention is a substrate made by treating a carrier with an amino group-containing silicon compound represented by the following general formula:
(RO)₃Si—(CH₂)ₖ—(C₆H₄)₁—(CH₂)ₘ—(NHCH₂CH₂)ₙ—NH₂
(wherein R is an alkyl group, and k is 1, 2, 3 . . . , l is 0, 1, 2, 3 . . . , m is 0, 1, 2, 3 . . . , n is 1, 2, 3 . . . ) to introduce an amino group onto a surface of the carrier. The upper limit of each of k, l and m is not particularly limited, but is preferably 3-20. The amino group-containing silicon compound represented by the above general formula has many substituents at its side chain. These substituents allow the protein to be immobilized through electrostatic interaction, hydrophobic interaction or the like. In the preferred embodiment of the invention, the protein is immobilized to the amino group of the above general formula through electrostatic interaction. From a viewpoint that the macromolecular protein is immobilized more stably, n in the above general formula is preferably 3-20.

The amino group-containing silicon compound for introducing the amino group is not particularly limited as far as it satisfies the above general formula. As the amino group-containing silicon compound may be mentioned, for example, (3-trimethoxysilylpropyl)-diethylene triamine and the like.

Also, the carrier to be provided with the amino group is not particularly limited, but can include an inorganic substrate such as a glass slide, a porous gel, a microwell plate, a silicon wafer or the like; and an organic substrate such as a polyester film, a polyethylene film or the like. Further, the shape of the carrier is not particularly limited, but may be, for example, a flat-plate shape such as a plate, a film or a sheet, and a three-dimensional shape such as a cube, a bar and a sphere.

The introduction of the amino group on the carrier is not particularly limited, but may be a so-called silane coupling. For example, a proper carrier is provided and immersed in a solution of the above amino group-containing silicon compound in a solvent such as toluene, methanol, ethanol or water and maintained at the solution temperature of 5-100° C. for about 1-12 hours, whereby the amino group-containing silicon compound is bonded to the carrier to introduce the amino group onto the surface of the carrier.

Then, the method for immobilizing proteins according to the present invention is described. That is, the method of the present invention is a method of immobilizing proteins to a substrate, characterized in that the substrate is made by treating a carrier with an amino group-containing silicon compound represented by the following general formula:
(RO)₃Si—(CH₂)ₖ—(C₆H₄)₁—(CH₂)ₘ—(NHCH₂CH₂)ₙ—NH₂
(wherein R is an alkyl group, and k is 1, 2, 3 . . . , l is 0, 1, 2, 3 . . . , m is 0, 1, 2, 3 . . . , n is 1, 2, 3 . . . ) to introduce an amino group onto a surface of the carrier, and immersed in a protein-containing buffer solution to immobilize the protein to the amino group through electrostatic interaction. Utilization of such an electrostatic interaction between the amino group and the protein allows the immobilization of the protein to be stabilized still more. In order to improve the stabilization, it is preferable to increase the number of the amino groups. The amino group-containing silicon compound is not particularly limited as far as it satisfies the above general formula. The same or different silicon compounds can be used in the immobilization of the protein.

The protein-containing buffer solution is preferable to contain a protein, a biopolymer or a biological sample (a cell, a microorganism, a tissue, an organ, etc.).

When l in the general formula is more than 1 (i.e. the amino compound has a phenyl group), the protein can be immobilized through hydrophobic interaction of the phenyl group. In this case, the force of immobilizing the proteins can be further improved by utilizing much hydrophobic interaction likewise the case that much hydrophobic interaction plays an important role even in the stabilization of primary structure of the protein.

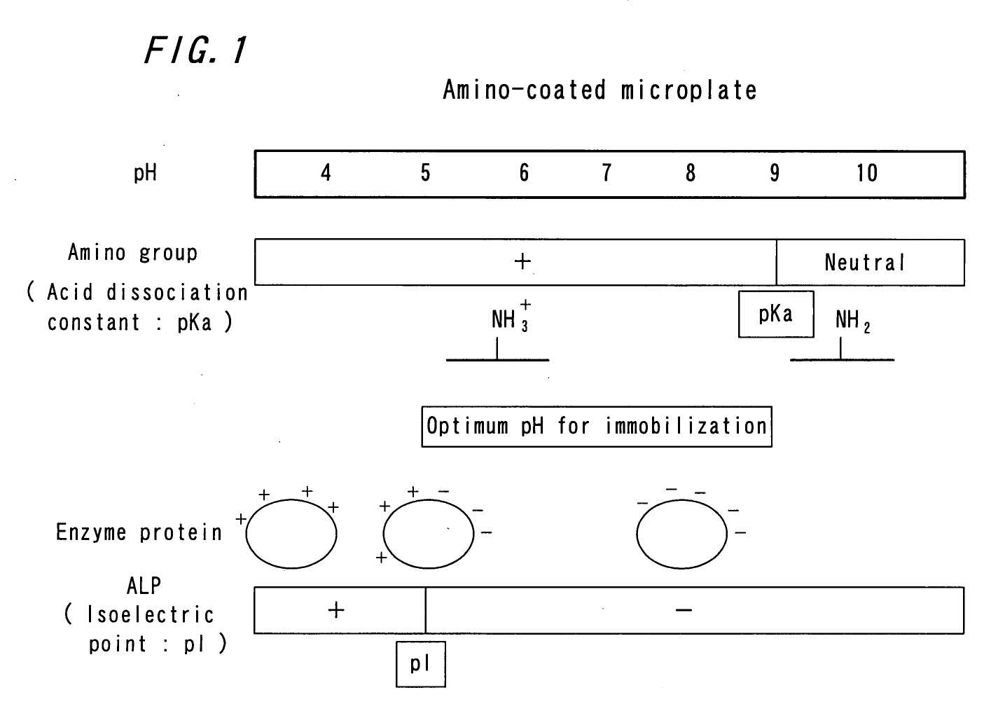

In case of immobilizing the proteins, it is preferable that when the isoelectric point of the protein is pI and the acid dissociation constant of the amino group is pKa, the pH of the protein-containing buffer solution satisfies a relation of pKa>pH>pI. In this case, the more stabilization of the immobilization through the stronger electrostatic interaction can be attained because, as shown in FIG. 1, when the value of pH is less than the value of acid dissociation constant pKa, the amino group on the substrate has positive charge, while when the value of pH is more than the value of the isoelectric point pI, the protein in the buffer solution has negative charge.

The following examples are given in illustration of the present invention and are not intended as limitations thereof.

### EXAMPLE 1-3

Preparation of an Aminated Plate

An aminated microtiterplate (pKa of amino group=8, hereinafter referred to as plate A) is prepared by treating a glass microtiterplate (96-well type, made by Nippon Sheet Glass Co., Ltd.,) with (3-trimethoxysilylpropyl)-diethylene triamine.

Preparation of Protein

There is provided an alkaline phosphatase (ALP, made by Wako Pure Chemical Industries, Ltd., Code No. 012-10691, pI=5).

Preparation of Buffer Solution

As a buffer solution having pH of 7.5 is prepared a buffer solution A (50 mM Tris-HCl buffer). As buffer solutions having pH of 6 and pH of 5 are prepared a buffer solution B and a buffer solution C (50 mM acetate buffer), respectively.

As a washing solution is prepared PBST (8 mM NaHPO₄, 2 mM KH₂PO₄, 0.145 M NaCl, 0.05% Tween 20).

The immobilization of ALP to the wells of the plate A is carried out as follows. 50 μl of ALP (100 μg/ml) dissolved in a buffer solution having the predetermined pH is applied to the wells, incubated at 37° C. for 6 hours, washed 9 times with phosphate buffer solution and 3 times with distilled water. The enzyme activity is evaluated using p-nitrophenyl phosphate (1 mM, 1 M tris-HCl, pH=8), which is a substrate of ALP. At first, 1 ml of p-nitrophenyl phosphate is applied to the wells, and the absorbance is measured by a spectrophotometer after 10 minutes. Then, in order to evaluate the capacity of repeat use, the wells are washed 5 times with the washing solution PBST, 3 times with 1 M NaCl and 2 times with distilled water, and thereafter the enzyme activity is measured. The results are shown in Table 1. As a result, when a relation of pKa>pH>pI is established among pKa of an amino group in the aminated plate, pI of the protein and pH of the buffer solution for immobilization, the effects of the invention are observed.

TABLE 1

| | | | Enzyme activity |
|---|---|---|---|
| | Plate | Buffer solution | (mAbs/min) |
| Example 1 | A | A | 100 |
| Example 2 | A | B | 90 |
| Example 3 | A | C | 40 |

### EXAMPLES 4-6

A plate B having an amino group is prepared in the same procedure as in Examples 1-3 except that (aminoethylaminomethyl)-phenetyltrimethoxysilane (k=2, l=61, m=1, n=1 in the above general formula) is used as an amino group-containing compound. The enzyme activity is examined using the plate B in the same manner as in Examples 1. The results are shown in Table 2.

TABLE 2

| | Plate | Buffer solution | Enzyme activity |
|---|---|---|---|
| Example 4 | B | A | 70 |
| Example 5 | B | B | 70 |
| Example 6 | B | C | 70 |

As seen from Table 2, the enzyme activity can be maintained by the hydrophobic action of the phenyl group without dependence on pH.

According to the present invention, therefore, it is recognized that the protein or the like can be strongly immobilized through electrostatic interaction but also the activity of the protein can be maintained.

The present invention can contribute to the utilization in the field related to biotechnology such as a protein array or the like.

1. A substrate having amino groups on a surface thereof, said substrate being prepared by treating a carrier with an amino group-containing silicon compound represented by the following general formula:
(RO)₃Si—(CH₂)ₖ—(C₆H₄)₁—(CH₂)ₘ—(NHCH₂CH₂)ₙ—NH₂
to introduce the amino groups onto a surface of the carrier, wherein R is an alkyl group, k is an integer ≧1, l is an integer ≧0, m is an integer ≧0, and n is an integer ≧1.

2. The substrate according to claim 1, wherein n is 3-20.

3. The substrate according to claim 1, wherein the amino group-containing silicon compound is (3-trimethoxysilylpropyl)-diethylene triamine.

4. A method of immobilizing a protein on a substrate having amino groups on a surface thereof, the method comprising contacting the substrate with a buffer solution comprising the protein, wherein the substrate is made by treating a carrier with an amino group-containing silicon compound represented by the following general formula:
(RO)₃Si—(CH₂)ₖ—(C₆H₄)₁—(CH₂)ₘ—(NHCH₂CH₂)ₙ—NH₂
to introduce amino groups onto a surface of the carrier, wherein R is an alkyl group, k is an integer ≧1, l is an integer ≧0, m is an integer ≧0, and n is an integer ≧1.

5. The method according to claim 4, wherein the buffer solution comprises a protein, a biopolymer or a biological sample.

6. The method according to claim 4, wherein the buffer solution comprises a biological sample derived from is at least one source selected from the group consisting of cells, microorganisms, tissues, and organs.

7. The method according to claim 4, wherein when l in the general formula is more than 1, the immobilization comprises a hydrophobic phenyl group interaction.

8. The method according to claim 4, wherein a pH of the protein-containing buffer solution satisfies a relation of pKa>pH>pI, wherein pI is an isoelectric point of the protein and pKa is an acid dissociation constant of the amino group.

9. The method according to claim 4, wherein the immobilization comprises electrostatic immobilization.

10. The method of claim 4, wherein said contacting comprises immersing the substrate in the buffer solution.
